(19) United States
(12) Patent Application Publication
(10) Pub. No.: US 2026/0224461 A1
(43) Pub. Date: Aug. 6, 2026

(54) Process for Curling Eyelashes Using a Composition Comprising Cysteine and an Organic Base

(71) Applicant: L'OREAL, PARIS (FR)

(72) Inventor: ALEXIS LIARD, SAINT-OUEN (FR)

(73) Assignee: L'OREAL, Paris (FR)

(21) Appl. No.: 19/152,981

(22) PCT Filed: Jan. 30, 2024
(86) PCT No.: PCT/EP2024/052128

§ 371 Date: Aug. 1, 2025

(30) Foreign Application Priority Data
Feb. 3, 2023 (FR) FR2301016

(51) Int. Cl. A61K 8/44; A45D 7/04; A45D 34/04; A61K 8/33; A61K 8/41; A61K 8/46; A61Q 1/10

(52) U.S. Cl. CPC A61K 8/447; A45D 7/04; A45D 34/045; A61K 8/33; A61K 8/41; A61K 8/463; A61Q 1/10; A61K 2800/87

(57) ABSTRACT

The present invention relates to a process for coating keratin fibres, in particular eyelashes, and more particularly for curling said keratin fibres, comprising the application to said keratin fibres of a composition comprising, in particular in a physiologically acceptable medium: a) at least 40.0% by weight of water relative to the total weight of the composition; and b) at least 1% by weight relative to the total weight of the composition of cysteine and/or a salt thereof; and c) at least one organic base; said composition having a pH of greater than 3.0; said process not comprising an oxidation step using an oxidizing agent other than air. The present invention also relates to a composition as defined previously, in which the organic base is monoethanolamine.

The present patent application relates to the field of making up keratin fibres, in particular the eyelashes.

In compositions for making up keratin surfaces such as the lower and/or upper eyelids, for instance eyeliners, consumers normally look for sheen, colour intensity and good wear properties, in particular water resistance.

In general, compositions dedicated to making up keratin fibres, such as the eyelashes, are targeted at densifying the thickness and the visual perception of the eyelashes or eyebrows and ultimately the look. However, another property very particularly desired by consumers is that of obtaining in a significant way a curving of said keratin fibres which is persistent to successive makeup removal operations. Water-resistant anhydrous mascaras do not make it possible to maintain the curvature of the eyelashes after several makeup removal operations.

In the salon, professionals use a makeup process inspired by techniques for the permanent waving of the hair which consists, in a first step, in applying, to the eyelashes, a reducing composition comprising a reducing agent (for example, ammonium thioglycolate and its derivatives, thiol compounds) which breaks the disulfide bridges of the keratin of the eyelashes and then, in a second step, in applying an oxidizing composition comprising at least one oxidizing agent, such as hydrogen peroxide, in order to re-establish the disulfide bridges of the keratin of the eyelashes, shaping the eyelashes and obtaining a durable curving. This process exhibits the disadvantages as being restricting, uncomfortable and not giving the consumer the possibility of easily carrying out this procedure herself at home.

The patent applications KR101823336 and KR101823401 have provided a process for the semipermanent curving of the eyelashes comprising the application, to the eyelashes, of a mascara comprising at least one reducing agent, such as cysteine, cysteamine, thiol compounds, sodium bisulfite, sodium metabisulfite, sodium hydrosulfite or superoxide dismutase, and at least one antioxidant, such as a compound based on phosphoric acid or a compound based on zinc, on gold, on tin or on silver, followed by washing and drying the eyelashes thus treated. This technique for the semipermanent curving of the eyelashes is not entirely satisfactory with regard to the significant curvature effect obtained and with regard to the persistence of the effect after a makeup removal operation.

Published patent application FR3114501A1 discloses a process for coating, and more particularly for curling eyelashes or eyebrows, comprising
at least a first step of applying a first composition (A) comprising, preferably in a physiologically acceptable medium, at least 40.0% of water, relative to the total weight of the composition, and at least 1.0% by weight, relative to the total weight of the composition, of cysteine and/or a salt thereof, said composition having a pH of greater than 8.0, and successively
at least a second step of applying, to said fibres treated with composition (A), at least a second composition (B), preferably comprising, in a physiologically acceptable medium:
i) an aqueous phase; and
ii) a fatty phase comprising at least one wax; and
iii) at least one anionic surfactant comprising at least one cationic counterion, preferably said anionic surfactant being in the form neutralized by said counterion;
iv) optionally at least one aqueous dispersion of film-forming polymer particles. This two-step gesture for curling the eyelashes can be felt by consumers to be constraining.

There is therefore still a need to find a new one-step process for making up the eyelashes which is easier, owing to the gesture involved, for consumers to use at home and produces an effective curling of said fibres that is persistent, without the constraints of the known processes of the prior art mentioned above, such as the use of an oxidizing agent other than air and/or of a shaping support.

Unexpectedly, the inventors have found that it is possible to achieve these objectives with a process for coating the eyelashes, and more particularly for curling the eyelashes, comprising at least the application to the eyelashes of a composition comprising, in particular in a physiologically acceptable medium:
a) at least 40.0% by weight of water relative to the total weight of the composition; and
b) at least 1.0% by weight relative to the total weight of the composition of cysteine and/or a salt thereof; and
c) at least monoethanolamine;
d) at least one anionic surfactant with an HLB of greater than or equal to 8.0 chosen from alkyl sulfates; and
e) at least one non-ionic surfactant with an HLB of greater than or equal to 8.0 chosen from polyoxyalkylenated alcohols;
said composition having a pH of greater than 8.0; said process not comprising an oxidation step using an oxidizing agent other than air.

The inventors have also unexpectedly discovered a new composition as defined above, in which the organic base is monoethanolamine.

This discovery forms the basis of the invention.

### SUBJECTS OF THE INVENTION

Thus, a first subject of the present invention is a process for coating the eyelashes, and more particularly for curling the eyelashes, comprising the application to the eyelashes of a composition comprising, in particular in a physiologically acceptable medium:
a) at least 40.0% by weight of water relative to the total weight of the composition; and
b) at least 1% by weight, relative to the total weight of the composition, of cysteine
c) at least monoethanolamine;
d) at least one anionic surfactant with an HLB of greater than or equal to 8.0 chosen from alkyl sulfates; and
e) at least one non-ionic surfactant with an HLB of greater than or equal to 8.0 chosen from polyoxyalkylenated alcohols;
said composition having a pH of greater than 3.0; said process not comprising an oxidation step using an oxidizing agent other than air.

### DEFINITIONS

In the context of the present invention, the term “keratin fibres” is understood to mean the eyelashes. For the purposes of the present invention, this term “keratin fibres” also extends to synthetic false eyelashes.

The term “cosmetically acceptable” means compatible with the keratin fibres, and the skin around the eye, which has a pleasant colour, odour and feel, and which does not cause any unacceptable discomfort (stinging or tautness) liable to discourage the consumer from using this composition.

### Cysteine

According to the present invention, the composition comprises at least 1.0% by weight of cysteine and/or of a salt thereof relative to the total weight of the composition.

Mention may more particularly be made, among the cosmetically acceptable salts of cysteine, of the hydrochlorides, hydrobromides, citrates, acetates and sulfates.

Preferably, the cysteine employed can be chosen from L-cysteine, D-cysteine or L,D-cysteine. Use will more particularly be made of L-cysteine or a salt thereof.

The cysteine and/or a salt thereof is preferably present at a concentration of from 1% to 20% by weight, more preferentially from 5% to 15% by weight relative to the total weight of the composition.

### Aqueous Phase

The composition according to the invention comprises at least 40.0% by weight of water relative to the total weight of the composition and has a pH of greater than 8.0.

The pH of the composition according to the invention preferably ranges from 8.5 to 10.0.

The alkaline pH is obtained by virtue of the presence of at least one organic base agent.

The organic base is monoethanolamine (MEA).

The pH of the composition may be adjusted with at least one acidifying agent and/or at least one buffering agent such as a phosphate buffer or a carbonate buffer.

The amount of water is at least 40.0% by weight, relative to the total weight of the composition. Preferentially, it ranges from 50% to 95% by weight and more preferentially from 55% to 90% by weight, relative to the total weight of the composition.

The water can be demineralized, or also a floral water, such as cornflower water, and/or a mineral water, such as Vittel water, Lucas water or water of La Roche-Posay water, and/or a spring water.

### Process for Curling Keratin Fibres

One subject of the present application is a process for curling the eyelashes, comprising at least the application to the eyelashes of a composition comprising, in particular in a physiologically acceptable medium:
a) at least 40.0% by weight of water relative to the total weight of the composition; and
b) at least 1.0% by weight relative to the total weight of the composition of cysteine and/or a salt thereof; and
c) at least one organic base;
said composition having a pH of greater than 3.0; said process not comprising an oxidation step using an oxidizing agent other than air.

According to a particular embodiment of said process, the application of the composition based on cysteine and/or a salt thereof is followed by a period in which said composition is left on the treated keratin fibres for a time ranging from 5 to 45 minutes. Preferably, after said leave-on period, the eyelashes are cleaned and then dried, for example by means of dry cotton wool at ambient temperature (20-25° C.).

According to a particularly preferential embodiment of this process, the cleaning of eyelashes treated with the composition based on cysteine and/or a salt thereof comprises the following steps:
a) a first eyelash makeup removal; then
b) bathing of the eyelashes from which the makeup was removed in step a), in a micellar solution for a period of time ranging from 1 to 5 minutes; then
c) a second eyelash makeup removal.

The makeup remover used in step a) can be a two-phase product such as La Roche Posay's commercial product Respectissime®.

The micellar solution used in step b) can be such as Garnier's commercial product All-in-1® micellar solution. It can be applied using cotton wool soaked in said product.

The makeup remover used in step c) may be a micellar solution.

The makeup removers are preferably applied to the keratin fibres by means of cotton wool soaked in the product.

The keratin fibre drying step can be carried out with dry cotton wool or in an oven.

### Cosmetic Additives

The composition may contain at least one conventional cosmetic additive chosen from hydrophilic gelling agents, fatty alcohols, preservatives, surfactants, fragrances, antioxidants, moisturizing agents such as polyols, for instance 1,3-propanediol, glycerol, pentylene glycol, caprylyl glycol, propylene glycol, ethylene glycol, 1,3-butylene glycol, dipropylene glycol, C₂-C₈ monoalcohols such as ethanol or isopropanol; and mixtures thereof.

Of course, a person skilled in the art will take care to choose the optional additional additives and/or their amounts such that the advantageous properties of the composition according to the invention are not, or not substantially, detrimentally affected by the envisaged addition.

### Hydrophilic Gelling Agent

According to a particular form of the invention, the composition according to the invention also comprises one or more hydrophilic gelling agent(s).

For the purposes of the present invention, the term “hydrophilic gelling agent” is understood to mean a compound capable of gelling the aqueous phase of the compositions according to the invention. More particularly, the function held by these hydrophilic gelling agents is to structure the aqueous phase in the composition. The gelling agent is advantageously soluble in the aqueous phase of the composition. The gelling agent which can be used according to the invention can in particular be characterized by its ability to form, in water, beyond a certain concentration C*, a gel characterized by oscillatory rheology (μ=1 Hz) by a yield point τ_{c} at least equal to 10 Pa. This concentration C* can vary widely according to the nature of the gelling agent under consideration.

The hydrophilic gelling agents are preferably chosen from non-ionic polysaccharides.

Non-ionic polysaccharides are carbohydrate macromolecules formed by the linking of a large number of hydrophilic elementary sugars (saccharides) bonded together via O-oside bonds.

They may be chosen from native polysaccharides, modified polysaccharides, and mixtures thereof.

The term “modified polysaccharide” means any chemically or enzymatically modified polysaccharide including at least one functional group.

The functional groups may be hydrocarbon-based groups (constituted essentially of carbon and hydrogen atoms) such as alkyl, alkenyl, aryl (i.e. phenyl) or aralkyl (i.e. benzyl) groups. The hydrocarbon-based groups may be unsubstituted, for example constituted of a simple alkyl chain or substituted with groups such as aromatic groups such as aryl (i.e. phenyl) or aralkyl (i.e. benzyl) groups or alternatively polar groups, for instance hydroxyl groups.

The optionally modified non-ionic polysaccharides that are suitable for use in the invention may be homopolysaccharides such as fructans, glucans, galactans and mannans or heteropolysaccharides such as hemicellulose. They may be starchy polysaccharides such as native or modified starches. The non-starchy polysaccharides may be chosen from polysaccharides produced by microorganisms; polysaccharides isolated from algae, and higher plant polysaccharides, such as homogeneous polysaccharides, in particular celluloses and derivatives thereof or fructoses, heterogeneous polysaccharides such as galactomannans, glucomannans and pectins, and derivatives thereof; and mixtures thereof. In particular, the polysaccharides may be chosen from fructans, glucans, amylose, amylopectin, glycogen, pullulan, dextrans, celluloses and derivatives thereof, in particular methylcelluloses, hydroxyalkylcelluloses and ethylhydroxyethylcelluloses, cetylhydroxyethylcelluloses, mannans, xylans, arabans, galactans, galacturonans, chitin, chitosans, glucuronoxylans, arabinoxylans, xyloglucans, glucomannans, arabinogalactans, agars, karaya gums (about 40% acid), locust bean gums, guar gums and non-ionic derivatives thereof, in particular hydroxypropyl guar, and biopolysaccharide gums of microbial origin, in particular scleroglucan gums. They are notably chosen from celluloses such as cetylhydroxyethylcelluloses; guar gums that are notably modified such as hydroxypropyl guars, agarose; pullulans, inulins and starches.

Preferably, use will be made of cellulose compounds, in particular hydroxyethylcellulose.

The hydrophilic gelling agent(s) are preferably present in the composition in a total content ranging from 0.1% to 10%, better still from 0.5% to 5% by weight, relative to the total weight of said composition.

### Surfactants

The composition according to the invention comprises at least one anionic surfactant with an HLB of greater than or equal to 8.0 chosen from alkyl sulfates and at least one non-ionic surfactant with an HLB of greater than or equal to 8.0 chosen from polyoxyalkylenated alcohols.

For the purposes of the present invention, the term “surfactant” is understood to mean an amphiphilic chemical compound, that is to say a compound exhibiting, in its structure, two parts of different polarity. Generally, one is lipophilic (soluble or dispersible in an oily phase). The other is hydrophilic (soluble or dispersible in water). Surfactants are characterized by the value of their HLB (hydrophilic-lipophilic balance), the HLB being the ratio of the hydrophilic part to the lipophilic part in the molecule. The term “HLB” is well known to a person skilled in the art and is described, for example, in “The HLB System. A Time-Saving Guide to Emulsifier Selection” (published by ICI Americas Inc.; 1984). The HLB of the surfactant(s) used according to the invention can be determined by the Griffin method.

The term “anionic surfactant” is understood to mean any negatively charged amphiphilic molecule.

The term “non-ionic surfactant” is understood to mean any amphiphilic molecule that is neutral, that is to say not negatively or positively charged.

According to a preferred embodiment of the invention, the surfactant(s) with an HLB of greater than or equal to 8.0 are present in a content ranging from 3.5% to 20.0% by weight, more preferentially from 4.0% to 20.0% by weight, better still from 4.5% to 15.0% by weight, even better still from 5.0% to 15.0% by weight relative to the total weight of the composition.

### a) Anionic Surfactants

The anionic surfactant(s) with an HLB of greater than or equal to 8.0 in accordance with the invention is (are) chosen from alkyl sulfates, and in particular alkyl ether sulfates, and alkylamido ether sulfates.

Use will more particularly be made of the anionic surfactant having the INCI name Ammonium Lauryl Sulfate, such as the commercial product Empicol AL 30/FL3® sold by Innospec Active Chemicals.

### b) Non-Ionic Surfactants

The non-ionic surfactant(s) with an HLB of greater than or equal to 8.0 in accordance with the invention is (are) preferably chosen from polyoxyalkylenated surfactants.

For the purposes of the present invention, a “polyoxyethylenated compound” is understood to mean any molecule comprising in its chemical structure at least one chain comprising oxyalkylene units, in particular —(OCH₂CH₂), oxyethylene and/or —(OCH₂CH₂CH₂)ₚ oxypropylene units.

The non-ionic surfactant(s) with an HLB value at 25° C. of greater than or equal to 8, preferably greater than or equal to 10, is (are) chosen from:
polyoxyalkylenated, in particular polyoxyethylenated and/or polyoxypropylenated,
alcohols that may comprise from 20 to 200 oxyethylene and/or oxypropylene units, preferably from 20 to 100 oxyethylene units, in particular polyethoxylated fatty alcohols, notably C₈-C₂₄ and preferably C₁₂-C₁₈ fatty alcohols, such as Steareth-20, for instance the product Brij 78® sold by Croda, and Ceteareth-30, for instance the product Eumulgin B 3® sold by BASF, or Oleth-30, for instance the product Eumulgin O 30® sold by BASF.

Use will more particularly be made of a polyoxyalkylenated alcohol, more particularly Oleth-30, for instance the product Eumulgin O 30® sold by BASF.

According to a particular embodiment, the composition of the invention comprises a mixture of Ammonium Lauryl Sulfate and Oleth-30.

### Fatty Alcohol

Preferentially, the compositions according to the invention also comprise at least one fatty alcohol. A composition can therefore comprise a single fatty alcohol according to the invention or several distinct fatty alcohols.

If several distinct fatty alcohols are present, they can be added separately during the preparation of the composition and the mixture thereof can then be formed in situ. They can also be used in the form of a mixture which is already commercially available and in which the weight proportion and the degree of purity of each of the fatty alcohols are controlled. In other words, the composition of these mixtures is faithfully reproducible, as opposed to mixtures of fatty alcohols generated via synthesis from starting materials derived from complex mixtures.

The fatty alcohol(s) is (are) in particular chosen from linear or branched, saturated or unsaturated C₁₄-C₃₀, preferably C₁₄-C₂₄, and even better still C₁₄-C₂₀, fatty alcohols.

The fatty alcohol(s) is (are) in particular chosen from linear and saturated C₁₄-C₃₀ fatty alcohols, preferably linear and saturated C₁₄-C₂₄ fatty alcohols, and better still linear and saturated C₁₄-C₂₀ fatty alcohols.

According to a particularly preferred embodiment, the fatty alcohol is in the form of a mixture of several different fatty alcohols, and is preferably a mixture of several linear and saturated C₁₄-C₃₀, better still C₁₄-C₂₄, even better still C₁₄-C₂₀, fatty alcohols.

Preferably, the fatty alcohol according to the invention is chosen from cetyl alcohol (C₁₆), stearyl alcohol (C₁₈) and mixtures thereof (also known as cetearyl alcohol). Preferentially, the fatty alcohol according to the invention is a mixture of cetyl alcohol and stearyl alcohol. Such a mixture is notably sold under the name Lannette O OR/MB® by BASF.

According to a preferred embodiment, the fatty alcohol is solid at ambient temperature.

The fatty alcohol is present in the compositions of the invention in amounts ranging from 1.0% to 20.0% by weight, relative to the total weight of the composition, preferably from 2.0% to 15.0% by weight and even more particularly from 3.0% to 10.0% by weight, relative to the total weight of the composition.

### Cosmetic Applications

The composition used according to the invention may be a curling product for the eyelashes, such as a mascara.

Such compositions are in particular prepared according to the general knowledge of a person skilled in the art.

### Packaging and Application Assembly or Kit

The present invention also relates to an assembly, or kit, for the packaging and application of a cosmetic composition for coating eyelashes, comprising:
a packaging device comprising said cosmetic composition for coating eyelashes as previously described,
an applicator for said composition.

According to another aspect, the invention also relates to a makeup assembly comprising:
i) an applicator suitable for drawing a line on the contour of the eye,
ii) a composition in accordance with the invention placed inside a container.

The container can delimit one or more compartment(s). The container can, for example, be in the form of a tube.

Such an applicator can be integral with a cap reversibly fitted to said container between a position of closure of said container and a makeup position.

In a variant, such an applicator can be irreversibly fitted to said container. Mention may be made, as examples of applicators, of those of felt or brush type which can be constituted of synthetic fibres.

It is understood that, in the context of the present invention, the percentages by weight given for a compound or a family of compounds are always expressed by weight relative to the total weight of the composition.

Throughout the patent application, the term “comprises one” should be understood as meaning “comprising at least one”, unless otherwise specified.

It is understood that the examples which follow are present by way of illustration and that they do not in any way limit the scope of the protection conferred by the present patent application.

### Example 1 (Invention) and Example 1a (Outside the Invention): Eyelash Curling Products

The following compositions were prepared:

| | | Ex. 1a |
|---|---|---|
| | | (outside |
| | Ex. 1 | the |
| EU INCI | (invention) | invention) |
| Cysteine | 10.0 | 10.0 |
| Sodium Hydroxide | | qs pH 9.5 |
| Ethanolamine | qs pH 9.5 | |
| Hydroxyethylcellulose | | |
| Phenoxyethanol | 0.5 | 0.5 |
| Caprylyl Glycol | 0.3 | 0.3 |
| Water | qs 100 | q.s. 100 |
| Cetearyl Alcohol | 10.2 | 10.2 |
| Ammonium Lauryl Sulfate | 2.4 | 2.4 |
| (Empicol AL 30 / FL3 ® - | (0.65 active | (0.65 active |
| Innospec Active Chemicals) | material) | material) |
| Oleth-30 | 1.8 | 1.8 |
| (Eumulgin O 30 ® - BASF) | | |

### Preparation Protocol

The compositions of Examples 1 and 1a were prepared using a Rayneri Turbotest VMI mixer equipped with a deflocculating rod, at a rotation speed of 3000 rpm for 1 minute.

### Tests for Measuring the Curvature of the Keratin Fibre

The formulae were applied to keratin fibres (human hair) in a proportion of 380 mg of formula per 8 mg of keratin fibres. The keratin fibres were then subjected to mechanical stress via a ramp with a radius of curvature of 5 mm and a tangent at the origin of 20°, for 30 minutes. The keratin fibres were subsequently rinsed with water, then subjected again to the same mechanical stress (ramp with a radius of curvature of 5 mm and a tangent at the origin of) 20° for 30 minutes. The keratin fibres thus treated were subsequently released from the mechanical stress and were then cleaned according to the following protocol:
1) cleaning using cotton wool soaked in Respectissime two-phase makeup remover from La Roche Posay then
2) immersion in a bath of micellar solution for 2 minutes, then
3) removal of makeup using cotton wool soaked in Garnier® All-in-1 micellar solution and
4) drying with dry cotton wool for 15 minutes.

The curvature is measured by measuring a height, between the base and the tip of the fibre. A fibre of which the height is 0 cm corresponds to a fibre that has not been shaped, while a fibre of which the height is close to 1 cm corresponds to a fibre that has been completely shaped.

### Results

| | | Ex. 1a |
|---|---|---|
| | | (outside |
| Measurement of the curvature | Ex. 1 | the |
| of the fibre | (invention) | invention) |
| Height (cm) | 0.9 | 0.6 |
| Standard deviation | 0.05 | 0.12 |

The results showed significantly that Example 1 of the invention comprising monoethanolamine a allowed the fibre to be completely shaped, unlike its counterpart 1a comprising an inorganic base (sodium hydroxide).

1. Process for coating eyelashes, and more particularly for curling eyelashes, comprising the application to said keratin fibres of a composition comprising, in particular in a physiologically acceptable medium:
a) at least 40.0% by weight of water relative to the total weight of the composition; and
b) at least 1% by weight relative to the total weight of the composition of cysteine and/or a salt thereof; and
c) at least monoethanolamine;
d) at least one anionic surfactant with an HLB of greater than or equal to 8.0 chosen from alkyl sulfates; and
e) at least one non-ionic surfactant with an HLB of greater than or equal to 8.0 chosen from polyoxyalkylenated alcohols;
said composition having a pH of greater than 3.0; said process not comprising an oxidation step using an oxidizing agent other than air.

2. Process according to claim 1, in which the cysteine and/or a salt thereof is chosen from L-cysteine, D-cysteine, L,D-cysteine, and a salt thereof.

3. Process according to claim 1, in which the cysteine and/or a salt thereof is present in a concentration of from 1% to 20% by weight relative to the total weight of the composition.

4. Process according to claim 1, in which the pH is from 8.5 to 10.0.

5. Process according to claim 1, in which water is present in the composition in an amount from 50% to 95% by weight relative to the total weight of the composition.

6. Process according to claim 1, in which the application of the composition to said keratin fibres is followed by a treatment period in which said composition is left on said keratin fibers for 5 to 45 minutes.

7. Process according to claim 6, in which, after said treatment period, said keratin fibres are cleaned and then dried.

8. Process according to claim 7, in which cleaning said keratin fibres comprises:
a) a first makeup removal; then
b) bathing of said keratin fibres in a micellar solution for a period of time from 1 to 5 minutes; then
c) a second makeup removal.

9. Process according to claim 1, in which the composition further comprises at least one cosmetic additive chosen from hydrophilic gelling agents, fatty alcohols, preservatives, surfactants, fragrances, antioxidants, moisturizers, and mixtures thereof.

10. Process according to claim 1, in which the composition further comprises at least one hydrophilic gelling agent.

11. Process according to claim 10, in which, in the composition, the hydrophilic gelling agent(s) is (are) present in a total content from 0.1% to 10% relative to the total weight of said composition.

12. Process according to claim 1, in which, in the composition, the at least one anionic surfactant with an HLB of greater than or equal to 8.0 and the at least one non-ionic surfactant with an HLB of greater than or equal to 8.0 are present in a content ranging from 3.5% to 20.0% by weight relative to the total weight of the composition.

13. Process according to claim 1, in which the at least one anionic surfactant with an HLB of greater than or equal to 8.0 is Ammonium Lauryl Sulfate and the at least one non-ionic surfactant with an HLB of greater than or equal to 8.0 is Oleth-30.

14. Assembly, or kit, for packaging and applying a cosmetic composition for coating eyelashes, comprising:
a packaging device comprising the composition as defined in claim 1;
an applicator for said composition.
